(19) United States
(12) Patent Application Publication
(10) Pub. No.: US 2025/0174302 A1
(43) Pub. Date: May 29, 2025

(54) METHODS FOR IDENTIFYING GENE INTERACTIONS, AND USES THEREOF

(71) Applicant: PANGEA BIOMED LTD., Tel Aviv (IL)

(72) Inventors: Isaac MEILIJSON, Tel Aviv (IL); Gal DINSTAG, Givataim (IL); Tuvik BEKER, Tel Aviv (IL)

(73) Assignee: PANGEA BIOMED LTD., Tel Aviv (IL)

(21) Appl. No.: 18/865,276

(22) PCT Filed: May 15, 2023
(86) PCT No.: PCT/IL2023/050497

§ 371 Date: Nov. 12, 2024

Related U.S. Application Data
(60) Provisional application No. 63/342,107, filed on May 15, 2022.

(51) Int. Cl. G16B 20/40; G16B 5/00; G16H 20/10

(52) U.S. Cl. CPC G16B 20/40; G16B 5/00; G16H 20/10

(57) ABSTRACT

Disclosed herein are methods for identifying a pair of genes comprising a synthetic lethality (SL), synthetic rescue (SR), or synthetic dosage lethality (SDL) interaction. The method is composed of two independent models. Also, disclosed herein are uses thereof.

### BACKGROUND OF THE INVENTION

Precision oncology has made significant advances in the last few years, mainly by targeting actionable mutations in cancer driver genes. However, the proportion of patients whose tumors can be targeted therapeutically remains limited. Recent studies have begun to explore the benefit of integrating tumor transcriptomics data to guide patient treatment, raising the need for new approaches to extract clinically actionable information from a tumor transcriptome.

Genetic interactions (GIs) have long been studied in model organisms as a means of identifying functional relationships among genes or their corresponding gene products, with the nature of these relationships depending on the types of interactions. Genetic interactions include pairs of genes comprising synthetic lethality (SL), synthetic rescue (SR), and synthetic dosage lethality (SDL) interactions. SL are genetic interactions in which co-inactivation of two genes is lethal to the cell, but individual inactivation of each gene is not. SR are genetic interactions in which following inactivation of the first gene, the cell either downregulates or upregulates the second gene in order to survive. SDL are genetic interactions in which the inactivation of the first gene coupled to the upregulation of a second gene is lethal to the cell.

Recent studies have begun to explore the utilization of transcriptomics data to guide cancer patients' treatment. These studies have reported encouraging results, testifying to the potential of such approaches to complement mutation panels and increase the likelihood that patients will benefit from genomics-guided, precision treatments. However, current approaches for utilizing tumor transcriptomics data to guide patient treatments are still of heuristic exploratory nature, raising the need for developing and testing new systematic approaches (see Lee, Ruppin et al., Nature Communications, 2018, incorporated herein by reference).

### SUMMARY OF THE INVENTION

The inventors have developed Enlight—a platform that identifies cancer vulnerabilities and uses them to predict response and resistance to oncological therapies based on multi-omics molecular data from the patient's tumor. Enlight uses an inference engine called SLIDE, which analyzes multiple sources of data (patient molecular data and health records, genetic screens and drug screens performed on cell lines, phylogenetic data, and more) in order to infer certain functional relationships between pairs of genes in the human genome, in the context of various cancer types. The present disclosure relates to certain improvements that were incorporated in SLIDE. Hereafter, reference may be made to SLIDE 1.0 and SLIDE 2.0, referring to the versions of SLIDE before and after the improvements described hereafter.

In some embodiments, the present disclosure relates to a method for identifying a pair of genes comprising a synthetic lethality (SL), synthetic rescue (SR), or synthetic dosage lethality (SDL) interaction, using a depletion model, the method implemented by a computer processor executing program instructions comprising:
a. Transforming expression data relating to each of two genes from a population, through ranking, thereby producing two uniform transformed distributions in the range [0, 1];
b. Calculating a resulting joint expression distribution for the gene pair, having uniform marginal distributions;
c. Identifying a parametric family of distributions comprising a shape parameter wherein said shape parameter determines the degree of corner depletion or enrichment for one or more of the corners in the joint distribution, and fitting said shape parameter to said joint expression data; and
d. Calculating a value of a best-fitting shape parameter as an indication of the genetic interaction between said two genes.

In some other embodiments, the present disclosure relates to a method for identifying a pair of genes comprising a synthetic lethality (SL), synthetic rescue (SR-DD or SR-DU), or synthetic dosage lethality (SDL) interaction, using a parametric survival model, the method implemented by a computer processor executing program instructions comprising:
a. Transforming expression data relating to each of two genes from a population, thereby producing two uniform transformed distributions in the range [0, 1];
b. Calculating a resulting joint expression distribution for the gene pair, having uniform marginal distributions;
c. Identifying a theoretical distribution function D that approximates the joint distribution of the transformed expression levels of said pair of genes;
d. Calculating a covariate value (c(p)) for a given patient in a population of patients (cohort P), by calculating a ratio of the density of said theoretical distribution function D at a joint expression (x,y) of said gene pair, to a maximal D density value or a minimal D density value, across a full joint distribution space; and
e. Assessing a correlation between (i) a set of covariates C:={c{p)|p in P} obtained in “c” for said patients of cohort P; and (ii) survival of the patients in said cohort, as an assessment of the strength of the corresponding genetic interaction between said gene pair.

In some other embodiments, the present disclosure relates to a method for creating genetic interaction graphs, the method implemented by a computer processor executing program instructions comprising
a. a method according to a method described above using a depletion model, a method described above using a parametric survival model, or a method described above using a depletion model and a parametric survival model;
b. including in the interaction graph all the genes belonging to gene pairs that passed the identification of step (a); and
c. marking each of said gene pairs with the type of interaction identified for it (namely, one of SL, SR-DD, SR-DU or SDL).

In some other embodiments, the present disclosure relates to a method of predicting responsiveness of a patient to a therapy targeting a set of target genes, the method implemented by a computer processor executing program instructions comprising:
a. identifying a genetic interaction graph according to the above;
b. incising from said genetic interaction graph of step (a) a sub-graph comprising said target genes and all other genes connected to said target genes (hereby “partner genes”); and
c. determining the activity of each said partner gene paired with one of the target genes, wherein low activity of multiple SL, SDL or SR-DU partner genes, and/or high activity of multiple SR-DD partner genes is indicative of high responsiveness to the therapy targeting said target genes;
   thereby predicting the responsiveness of the patient to the therapy.

In some other embodiments, the present disclosure relates to a method of stratifying a population of patients according to the responsiveness to a therapy targeting a set of target genes, the method implemented by a computer processor executing program instructions comprising:
a. predicting responsiveness of each patient of the population to the therapy according to the method described above; and
b. stratifying said population of patients according to their responsiveness to the therapy.

In some other embodiments, the present disclosure relates to a method for identifying a drug target for a disease, wherein the disease is associated with the inactivation of a single target gene, the method implemented by a computer processor executing program instructions comprising:
a. identifying a genetic interaction graph spanning the target genes according to the method described above;
b. incising from said genetic interaction graph of step (a) a sub-network comprising said target gene and all other genes connected to said target gene;
c. for each said target gene, stratifying designated patient population cohort P, according to the method described above, based on predicted response to inhibition of said target gene; and
d. identifying the most attractive potential drug targets, as those target genes for which a significant sub-population of patients is expected to respond to target inhibition.

In some other embodiments, the present disclosure relates to a method for identifying synergistic drugs for treating a disease, the method implemented by a computer processor executing program instructions comprising:
a. identifying a genetic interaction graph according to the method described above;
b. Incising pairs from said genetic interaction graph for which both genes are known drug targets; and
c. Prioritizing said pairs found in step (b) according to in vitro experiments.

In some other embodiments, the present disclosure relates to a method for designing a clinical trial for a therapy, the method implemented by a computer processor executing program instructions comprising:
a. stratifying a population of patients according to the method described above; and
b. including in said clinical trial only patients predicted to be responsive to the therapy.

In some other embodiments, the present disclosure relates to a method for prioritizing in vitro models for drug development, the method implemented by a computer processor executing program instructions comprising: the method according to the method described above, where the stratification is done for cell-lines instead of human patients.

In some other embodiments, the present disclosure relates to a method for repurposing existing drugs to novel indications, wherein a given drug targets a given gene or a given set of genes, the method implemented by a computer processor executing program instructions comprising:
a. Stratifying patient cohorts from different cancer types according to the method described above; and
b. Identifying cohorts with maximal number of predicted responders.

In some other embodiments, the present disclosure relates to a method for expanding the indications of a drug targeting a gene or a set of genes, the method implemented by a computer processor executing program instructions comprising:
a. providing a cohort of patients having a medical condition, wherein said condition is not indicated to said drug;
b. predicting the responsiveness of each patient of said cohort to a therapy comprising administering said drug, according to the method described above;
   wherein high responsiveness to said therapy indicates that said drug can be indicated to said medical condition.

In some other embodiments, the present disclosure relates to a method for expanding the indications of a drug targeting a gene or a set of genes, the method implemented by a computer processor executing program instructions comprising:
a. providing a group of patients having a medical condition, wherein said condition is not indicated to said drug;
b. predicting the responsiveness of each patient to a therapy comprising administering said drug, according to the method described above;
c. stratifying said patients according to their responsiveness to said therapy;
d. identifying a cohort with the maximal number of predicted responders;
   wherein high responsiveness to said therapy in said cohort indicates that said drug can be indicated to said medical condition for patients belonging to said cohort.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates a detailed description of the depletion test algorithm. The parabola-shaped function describes the trajectory of summed log-likelihood values as a function of theta for an example gene pair. The vertical dashed line marks θ\hat{ };

FIG. 2 illustrates that the Gumbel method identifies known GIs to a larger extent than the hypergeometric method, throughout the wide threshold range;

FIG. 3 illustrates detailed description of the parametric survival test algorithm;

FIG. 4 illustrates that the performance of the parametric survival test of SLIDE 2.0 far exceeds the performance of the cox-based model in identifying known GIs;

FIG. 5 illustrates that the new parametric model is indeed more robust to perturbations, with far lower dispersion than in the cox model; and

FIG. 6 illustrates the performance of response prediction for several patient groups with SLIDE 1 and SLIDE 2. Each category on the X axis represents a different group of patients who received an indicated treatment (EGFRi=EGFR inhibitor). The Y axis denotes the precision (Positive Predictive Value) at 50% recall (i.e., for a threshold that identifies 50% of the responders). The rightmost category is for Bevacizumab, a drug for which the interaction network in SLIDE 1.0 was empty. Hence, only results for SLIDE 2.0 are presented.

FIG. 7 illustrates ENLIGHT's ability to stratify patients for therapy. FIG. 7A shows the OR for response of ENLIGHT-matched cases in the 21 evaluation cohorts (OR values appear on top of each bar; all eight patients predicted to respond in the bevacizumab2 cohort responded to the treatment, resulting in an infinite OR), along with the OR for the aggregation of all cohorts and aggregation based on therapeutic class. Sample sizes are denoted in parentheses. Cohorts for which OR is significantly larger than 1 according to Fisher's exact test are denoted with asterisks. “Anti-PD1” encompasses three different drugs (nivolumab, pembrolizumab, and durvalumab). Vertical error bars in the “All” bar denotes 95% confidence interval for the OR. FIG. 7B is analogous to FIG. 7A but presents the sensitivity and PPV of ENLIGHT-matched cases versus the overall response rate for the evaluation cohorts and their aggregations. Significant differences between PPV and response rate according to the one-sided proportion test are denoted with asterisks. FIG. 7C shows that in the WINTHER trial, responders (orange) have significantly higher EMS than non-responders (blue); the p value was calculated using a one-sided Mann-Whitney test. 95% confidence interval for the OR is denoted in brackets. The horizontal line marks the decision threshold (0.54). FIG. 7D shows the sensitivity and PPV of ENLIGHT-matched cases versus overall response rate in the WINTHER trial. p value was calculated according to the one-sided proportion test. †Patients in these cohorts received a combination of targeted and chemotherapy; *p<0.1, **p<0.05. FIG. 7E shows the analysis of the 24 patients that were treated with a combination of ENLIGHT-analyzable drugs in the WINTHER trial. Responders have significantly higher EMS than non-responders, p-value is based on one sided Mann-Whitney test. The horizontal line marks the decision threshold for considering a treatment as favorable for a patient (EMS≥0.54). OR: odds ratio. FIG. 7F illustrates a heatmap showing the EMS for the 96 patients analyzed in the WINTHER trial (columns) and all ENLIGHT analyzable drugs given in the trial (rows). The ‘Winther’ row shows the EMS for the treatment regimen given in the trial. Color designates EMS, with red colors corresponding to ENLIGHET-matched treatments (EMS≥0.54). Black boxes indicate the drugs that were given to each patient. ‘Other treatments’: non-analyzable drugs, i.e., chemotherapy or hormonal therapy. The cancer type of each sample is color-coded at the top of the heatmap.

FIG. 8 illustrates that ENLIGHT can facilitate the exclusion of non-responding patients in clinical trials. Each of the three columns depicts ENLIGHT's results on the aggregate of all evaluation cohorts from a given therapeutic class. Panels on the top row display the NPV (percentage of true non-responders out of those predicted as non-responders) as a function of the percentage of patients excluded. The horizontal line denotes the actual percentage of non-responders in the corresponding aggregate cohort (i.e., the NPV expected by chance). Panels on the bottom row display the response rate among the remaining patients (y axis) after excluding a certain percentage of the patients (x axis). The horizontal line denotes the overall response rate in the aggregate cohort. The dotted-dashed line represents the upper bound on the response rate, achieved by the “all knowing” optimal classifier excluding only true non-responders.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present methods may be understood more readily by reference to the following detailed description which forms a part of this disclosure. It is to be understood that this disclosure is not limited to the specific methods or parameters described and/or shown herein, and that the terminology used herein is for the purpose of describing particular embodiments by way example only and is not intended to be limiting of the claimed disclosure. Similarly, it is to be understood that the embodiments disclosed herein are combinable.

Unless otherwise defined herein, scientific, and technical terms used in connection with the present application shall have the meaning that are commonly understood by those of ordinary skilled in the art. Further, unless otherwise required by context, singular terms shall include pluralities and plural terms shall include the singular.

A skilled artisan would appreciate that the term “comprising” encompasses inclusion of the recited elements, but not excluding others which may be optimal. For example, “comprising calculating a Gumbel copula statistical model” can comprise additional elements in the calculation.

In some embodiments, the present disclosure relates to a method for identifying a pair of genes comprising a synthetic lethality (SL), synthetic rescue (SR), or synthetic dosage lethality (SDL) interaction. The method implemented by a computer processor executing program instructions is comprised of two independent models.

In some embodiments, the present disclosure related to a method for expanding the indication of an existing drug. Said method, sometimes called “label expansion” aims to find alternative therapeutic applications, or indications for an existing drug target.

### 1. Depletion Model

In some embodiments, the present disclosure relates to a method for identifying a pair of genes comprising a synthetic lethality (SL), synthetic rescue (SR), or synthetic dosage lethality (SDL) interaction, using a depletion model, the method implemented by a computer processor executing program instructions comprising:
a. Transforming expression data relating to each of two genes from a population, through ranking, thereby producing two uniform transformed distributions in the range [0, 1];
b. Calculating a resulting joint expression distribution for the gene pair, having uniform marginal distributions;
c. Identifying a parametric family of distributions comprising a shape parameter wherein said shape parameter determines the degree of corner depletion or enrichment for one or more of the corners in the joint distribution, and fitting said shape parameter to said joint expression data; and
d. Calculating a value of a best-fitting shape parameter as an indication of the genetic interaction between said two genes.

A skilled artisan would appreciate the term “shape parameter” as a kind of numerical parameter of a parametric family of probability distributions. Specifically, a shape parameter is any parameter of a probability distribution, which affects the shape of the distribution rather than simply shifting it or stretching/shrinking it.

A skilled practitioner in the art would appreciate that the basic principle of synthetic lethality (SL) dictates the following: if two genes are synthetically lethal, their co-inactivation is lethal and thus it is unlikely to observe simultaneous inactivation of the two genes in the same cell, or in a bulk sample from a cell culture. The detailed description focuses on synthetic lethality, although a similar principle applies to other genetic interactions by using different combinations of activation states of gene pairs.

A skilled practitioner would appreciate that the basic principle for synthetic rescue (SR) dictates the following: a functional interaction between two genes or nucleic acid sequences in which a change in the activity of a vulnerable gene (which may be a target of a cancer drug) is lethal, but the subsequent altered activity of its partner (rescuer gene) restores cell viability.

In one embodiment, the synthetic rescue (SR) comprises synthetic rescue DD (SR-DD) or synthetic rescue DU (SR-DU). In another embodiment, the synthetic rescue (SR) comprises synthetic rescue DD (SR-DD). In another embodiment, the synthetic rescue (SR) comprises synthetic rescue DU (SR-DU).

A skilled practitioner in the art would appreciate that the basic principle of synthetic rescue DU (SR-DU) is where the down-regulation of a vulnerable gene is lethal but the cell is rescued by the upregulation of its rescuer partner. A skilled practitioner in the art would appreciate that the basic principle of synthetic rescue DD (SR-DD) is where the downregulation of a vulnerable gene is rescued by the downregulation of a rescuer gene.

A skilled practitioner in the art would appreciate that the basic principle of synthetic dosage lethality (SDL) is the genetic interactions in which the inactivation of the first gene coupled to the upregulation of a second gene is lethal to the cell.

In one embodiment, the present disclosure relates to a method wherein for identifying a pair of genes comprising a synthetic lethality (SL) interaction, the shape parameter would measure depletion in the lower left corner of said joint distribution. In another embodiment, the present disclosure relates to a method wherein for identifying a pair of genes comprising a synthetic rescue DD (SR-DD) interaction, the shape parameter would measure enrichment in the lower left corner of said joint distribution. In another embodiment, the present disclosure relates to a method wherein for identifying a pair of genes comprising a synthetic rescue DU (SR-DU) interaction, the shape parameter would measure enrichment in the upper left corner of said joint distribution. In another embodiment, the present disclosure relates to a method wherein for identifying a pair of genes comprising a synthetic dosage lethality (SDL) interaction, the shape parameter would measure depletion in the upper left corner of said joint distribution.

One of the statistical tests that can be used to infer genetic interactions stems from this phenomenon and will be called herein the “depletion test”. The depletion test identifies gene pairs for which the simultaneous inactivation of pairs in a population is depleted, i.e., the number of cases in a population where a gene pair is simultaneously inactive is lower than the number expected to observe by mere chance. There are many ways to model such depletion in statistical means. One such method is implemented in the ISLE algorithm as described in Lee, Ruppin et al., Nature Communications, 2018, (incorporated herein by reference) and makes use of the hyper-geometric distribution test. However, the application of this test in the above-mentioned algorithms forces certain marginal expression distributions on the two genes, which violates the preliminary assumptions of the hyper-geometric test. In addition, the former method discretized the data in a way that eliminates most of the variability incorporated in the data, causing loss of useful information.

In this disclosure, a different statistical model is introduced, which is used in SLIDE 2.0 and overcomes these drawbacks. The model uses the Gumbel copula: a statistical model that describes the dependency structure between two random variables through their individual cumulative distributions, with the constraint that all marginal distributions are uniform. Gumbel copula describes the joint distribution of a random variable pair as a function with high density on the primary diagonal (i.e. the straight line where x=y) and exponential decay in density away from the primary diagonal. The strength of dependency is described by the parameter theta. When theta=1, the two random variables are completely independent, i.e., the joint distribution is uniform, and the primary diagonal has the same density as all other areas. As theta grows, higher density is placed on the primary diagonal of the joint distribution, and the probability of an object on the joint distribution to be away from the diagonal decreases exponentially with distance. One explanation for joint distribution characterized by high theta is a distribution with depletion in one or two of the corners: if the distribution is indeed depleted in these areas, most values will gather around the primary diagonal, which fits a Gumbel distribution with high theta parameter. However, the current disclosure relates to depletion around low values for both genes, while the original Gumbel distribution also accounts for depletion in the area of high values for both genes.

To correct this, introduced herein is a modification to the original Gumbel distribution (termed “modified Gumbel”): instead of symmetric decay in probability away from the diagonal, the upper triangle of the joint distribution was modified to have constant probability. This gives a new model which, for high theta value, specifically fits a distribution which is depleted in the lower part of the joint distribution: the highest probability lies on the primary diagonal, decays exponentially to the direction of low values for both genes, and remains constant above the diagonal. Finally, the modified Gumbel distribution is mirrored horizontally through the transformation

$$x ⇒ 1 - x + \frac{1}{N}$$

(N being the number of samples), to achieve the desired distribution that measures depletion near the origin, rather than around the corner (1,0).

In another embodiment, the parametric family of distributions comprises Gumbel copulas and said shape parameter comprises a parameter theta of the copula.

In another embodiment, the present disclosure relates to a method implemented by a computer processor executing program instructions comprising
a. selecting a pair of genes from genomic data across a population of N samples;
b. building a distribution for each of said pair of genes across the population;
c. for each of said pair of genes, assigning ranks corresponding to said distribution, the ranks being evenly distributed between 0 and 1, i.e., ranking the distribution of each gene and dividing by the number of samples to obtain values in the range [0,1];
d. obtaining data by mirroring said distribution horizontally, by transforming the ranks for one of the genes by:

$$x ⇒ 1 - x + \frac{1}{N} .$$

e. calculating a theta value which maximizes a likelihood of a Gumbel model of said data obtained in (d) for the gene pair:

$${θ}^{⋀} = {argmax}_{θ} {\sum }_{i = 1}^{n} log [ {Gumbel}_{θ} ( {g}_{1 i} , {g}_{2 i} ) ] .$$

A skilled practitioner in the field would appreciate that Theta can accept any value in the range [1, ∞]. In one extreme case, theta=1, in which case the marginal uniform distributions are completely independent as mentioned above. At the other extreme, very high theta values imply near-perfect negative correlation between the marginal distributions.

### 2. Parametric Survival Model

Survival of cancer patients is affected by diverse factors—some related to the specific cancer type, organ, and other histological characteristics, some related to the physiological background of the patient such as comorbidities, and some related to the course of treatment. These factors are relatively easy to assess or measure. A factor with even more profound effect on the survival of a patient, but one that is much more difficult to measure or even understand, is the molecular and genetic makeup of the tumor. Under this criterion are DNA mutations potentially causing the cancerous process, the microenvironment of the tumor, and many other biological features that encompass the state and shape of the tumor. Understanding the biological makeup of the tumor is key for identifying appropriate treatments. SLIDE algorithm uses genomic analysis of tens of thousands of cancer patients to find patterns that will lead to insights on the cancerous processes, specifically ones that can be harnessed in order to identify good existing treatments or discover novel ones.

The goal of the parametric survival model is to identify Genetic Interactions (GIs) that possess clinical impact under the following premise: if a GI between two genes exists, this interaction should leave a clinical footprint in the form of survival impact on patients. For example, if two genes are synthetically lethal, patients in whose tumors the genes are simultaneously inactive should have better survival than others, because the synthetic lethal interaction would lead to cancer cell death in those individuals. Moreover, patient genomics should suffice to uncover those with active synthetic lethal pairs (i.e. patients with simultaneous inactivation) and by linking the genomics with survival data, one can identify a pattern of favorable survival towards patients with active synthetic lethal pairs. Thus, by screening many putative pairs, those that show an association between the joint activation state and survival on a cohort of patients are more likely to have a clinically significant GI. Such screening is applied as part of the SLIDE algorithm and termed the survival test.

The SLIDE 1.0 survival test analyses individual interactions, one at a time, in the following manner: first, a patient population on which to test the interaction is determined. Next, the genomic data of the specific genes for each patient (e.g., the mRNA expressions or copy number variations) are used to categorize the patients into two groups: those with simultaneous inactivation of the two genes and those without such co-inactivation. Finally, a cox proportional hazard model is fitted to the simultaneous inactivation state of the patients to assess whether this state is associated with the survival of the patients. The analysis also controls for possible confounding factors such as age, gender, cancer stage and tumor origin. There are three main drawbacks in this model: 1. The cox proportional hazard test is a semi-parametric model which has weaker statistical power compared to full parametric models; 2. The existing test was shown to be sensitive to small perturbations in the data; 3. The binary categorization of patients leads to significant loss of information.

Described herein, a new statistical model for the survival test which aims to solve these three issues, SLIDE 2.0. The statistical model is based on the exponential model for survival: in its basic form, it assumes that the time to failure (or an “event”) depends exponentially on a linear combination of factors, i.e., Survival(β, x, t)=e^{-x}^{^{T}}^{βt} where x is a series of factors impacting survival, β is a vector of coefficients which represent the magnitude of impact each factor has on survival and t represents the time at which the event occurs. This model if fully parametric and the coefficients β can be estimated using numerical methods such as the Newton-Raphson method.

In addition to the change in the statistical model, described herein, a new way to quantify the state of gene pair co-activity. As described above, the former method rigidly categorized all patients into one of two groups, depending on whether or not a given pair of genes are simultaneously inactive, using some cutoff of inactivation on the genomic data. Here, introduced for the first time, is a new quantification that is based on the joint distribution calculated in the depletion test. Specifically, a continuous variable was calculated for each patient that stems from the position of its genomics on the joint density function of the fitted Gumbel model. This way, the survival depends gradually on the joint activation state of the gene pair rather than on a binary random variable.

In some embodiments, the present disclosure relates to a method for identifying a pair of genes comprising a synthetic lethality (SL), synthetic rescue (SR-DD or SR-DU), or synthetic dosage lethality (SDL) interaction, using a parametric survival model, the method implemented by a computer processor executing program instructions comprising:
a. Transforming expression data relating to each of two genes from a population, thereby producing two uniform transformed distributions in the range [0, 1];
b. Calculating a resulting joint expression distribution for the gene pair, having uniform marginal distributions;
c. Identifying a theoretical distribution function D that approximates the joint distribution of the transformed expression levels of said pair of genes;
d. Calculating a covariate value (c(p)) for a given patient in a population of patients (cohort P), by calculating a ratio of the density of said theoretical distribution function D at joint expression values (x,y) of said gene pair, to a maximal D density value or a minimal D density value, across a full joint distribution space; and
e. Assessing a correlation between (i) a set of covariates C:={c{p)|p in P} obtained in “c” for said patients of cohort P; and (ii) survival of the patients in said cohort, as an assessment of the strength of the corresponding genetic interaction between said gene pair.

A skilled artisan would appreciate the term “covariance” as a measure of the joint variability of two random variables. If the greater values of one variable mainly correspond with the greater values of the other variable, and the same holds for the lesser values (that is, the variables tend to show similar behavior), the covariance is positive. In the opposite case, when the greater values of one variable mainly correspond to the lesser values of the other, (that is, the variables tend to show opposite behavior), the covariance is negative. The sign of the covariance therefore shows the tendency in the linear relationship between the variables.

In one embodiment, the distribution function D is identified according to the method described above (depletion mode). In another embodiment, the distribution function comprises Gumbel copula statistical model.

In one embodiment, the method implemented by a computer processor executing program instructions comprises:
a. Selecting a pair of genes from genomic data across a population of N samples;
b. Building a distribution for each of said pair of genes in the population;
c. For each gene, assigning ranks corresponding to said distribution, the ranks being evenly distributed between 0 and 1, i.e., ranking the distribution of each gene and dividing by the number of samples to obtain values in the range [0,1];
d. Obtaining data by mirroring said distribution horizontally, by transforming the ranks for one of the genes by:

$$x ⇒ 1 - x + \frac{1}{N} .$$

e. Calculating theta θ according to the method described above;
f. For each patient p from said sample, calculating a covariate Cₚ according to:

$${C}_{p} = log ( \frac{max ( {Gumbel}_{θ} )}{{Gumbel}_{θ} ( {g}_{1 , p} , {g}_{2 , p} )} )$$

Where g_{i,j} denotes the expression of gene gi in patient j
g. Calculating likelihood of the parametric survival model according to:

$$L ( C , t , β ) = {\prod }_{i ∈ OBS} S ( {t}_{i} , {c}_{i} , β ) * λ ( {t}_{i} , {c}_{i} , β ) {\prod }_{i ∉ OBS} S ( {t}_{i} , {c}_{i} , β ) \\ {\prod }_{i ∈ OBS} {e}^{{C}^{T} β} {e}^{- {e}^{{C}^{T} {β}_{t}}} {\prod }_{i ∉ OBS} {e}^{- {e}^{{C}^{T} {β}_{t}}} ,$$

where C,t are the vectors of cₚ values and event times for all the patients in said population respectively, and β is the coefficient associated with C.

In one embodiment, the present disclosure relates to a method implemented by a computer processor executing program instructions using a depletion model and/or the method using a parametric survival mode wherein the expression data comprises single-cell data, shRNA/sgRNA screens, CRISPR single gene knockout, drug screens, patient data from the Cancer Genome Atlas (TCGA) or any combination thereof. In one embodiment, the expression data comprises single-cell data. In another embodiment, the expression data comprises shRNA/sgRNA screens. In another embodiment, the expression data comprises CRISPR single gene knockout. In another embodiment, the expression data comprises drug screens. In another embodiment, the expression data comprises patient data from the Cancer Genome Atlas (TCGA).

In one embodiment, the present disclosure relates to the method implemented by a computer processor executing program instructions using a depletion model and/or the method using a parametric survival mode wherein the distribution is measured directly through protein expression data, deduced from measurements of methylation, silencing DNA mutations, mRNA expression, mRNA copy number variation or any combination thereof. In one embodiment, the distribution is measured directly through protein expression data. In another embodiment, the distribution is deduced from measurements of methylation. In another embodiment, the distribution is deduced from silencing DNA mutations. In another embodiment, the distribution is deduced from mRNA expression. In another embodiment, the distribution is deduced from mRNA copy number variation.

In one embodiment, the present disclosure relates to the method implemented by a computer processor executing program instructions using a depletion model and/or the method using a parametric survival mode wherein the population comprises human cell lines, patients, or combination thereof, in one embodiment, the population comprises human cell lines. In another embodiment, the population comprises patients. In another embodiment, the population comprises human cell lines and patients.

In one embodiment, the present disclosure relates to a method for identifying a pair of genes comprising a synthetic lethality (SL), synthetic rescue (SR-DD or SR-DU), or synthetic dosage lethality (SDL) interaction, the method implemented by a computer processor executing program instructions comprising combining a depletion model according to the above and a parametric survival model according to the above.

### 3. Use

In one embodiment, the present disclosure relates to a method for creating genetic interaction graphs, the method implemented by a computer processor executing program instructions comprising:
a. a method according to the method described above using depletion model, a method described above using parametric survival model, or a method described above using depletion model and parametric survival model;
b. including in the interaction graph all the genes belonging to gene pairs that passed the identification of step (a); and
c. marking each of said gene pairs with the type of interaction identified for it (namely, one of SL, SR-DD, SR-DU or SDL).

In one embodiment, the type of interaction is SL. In another embodiment, the type of interaction is SR-DD. In another embodiment, the type of interaction is SR-DU. In another embodiment, the type of interaction is SDL.

In one embodiment, the present disclosure relates to a method of predicting responsiveness of a patient to a therapy targeting a set of target genes, the method implemented by a computer processor executing program instructions comprising:
a. identifying a genetic interaction graph according to the method described above;
b. incising from said genetic interaction graph of step (a) a sub-network comprising said target genes and all other genes connected to said target genes (hereby “partner genes”); and
c. determining the activity of each said partner gene paired with one of the target genes, wherein low activity of multiple SL, SDL or SR-DU partner genes, and/or high activity of multiple SR-DD partner genes is indicative of high responsiveness to the therapy targeting said target genes;
   thereby predicting the responsiveness of the patient to the therapy.

In one embodiment, the present disclosure relates to a method of stratifying a population of patients according to the responsiveness to a therapy targeting a set of target genes, the method implemented by a computer processor executing program instructions comprising:
a. predicting responsiveness of each patient of the population to the therapy according to the method described in the method described above; and
b. stratifying said population of patients according to their responsiveness to the therapy.

In one embodiment, the patients are diagnosed with cancer. In another embodiment, the cancer is solid cancer or hematological cancer. In another embodiment, the solid cancer is selected from breast cancer, gastrointestinal cancer, skin cancer, lung cancer, brain cancer, bladder cancer, cervical cancer, endometrial cancer, kidney cancer, lip cancer, oral cancer, liver cancer, ovarian cancer, pancreatic cancer, prostate cancer, thyroid cancer or any combination thereof, in another embodiment, the hematological cancer is selected from leukemia, Non-Hodgkin lymphoma, Hodgkin lymphoma, Multiple myeloma or any combination thereof.

In one embodiment, the therapy is an anti cancer therapy. In another embodiment, the anticancer therapy is Bevacizumab. In another embodiment, the anticancer therapy is Bortizomib. In another embodiment, the anticancer therapy is Everolimus. In another embodiment, the anticancer therapy is Sorafenib. In another embodiment, the anticancer therapy is Tipifamib. In another embodiment, the anticancer therapy is an EGFR inhibitor. In another embodiment, the anticancer therapy is a BRAF inhibitor. In another embodiment, the anticancer therapy is an anti PD1. In another embodiment, the anticancer therapy is an anti PDL1. In another embodiment, the anticancer therapy is a BRAF inhibitor. In another embodiment, the anticancer therapy is an EGFR inhibitor. In another embodiment, the anticancer therapy is chemotherapy therapy.

As used herein, an “inhibitor” of a given protein refers to modulatory molecules or compounds that, e.g., bind to, partially or totally block activity, decrease, prevent, delay activation, inactivate, desensitize, or down regulate the activity or expression of the given protein, or downstream molecules regulated by such a protein. Inhibitors can include siRNA or antisense RNA, genetically modified versions of the protein, e.g., versions with altered activity, as well as naturally occurring and synthetic antagonists, antibodies, small chemical molecules and the like.

In one embodiment, the present disclosure relates to a method of identifying a drug target for a disease, wherein the disease is associated with the inactivation of a single target gene, the method implemented by a computer processor executing program instructions comprising:
a. identifying a genetic interaction graph spanning the target genes according to the method described above;
b. incising from said genetic interaction graph of step (a) a sub-network comprising said target gene and all other genes connected to said target gene;
c. for each said target gene, stratifying designated patient population cohort P, according to the method described above, based on predicted response to inhibition of said target gene; and
d. identifying the most attractive potential drug targets, as those target genes for which a significant sub-population of patients is expected to respond to target inhibition.

In one embodiment, the present disclosure relates to a method for identifying synergistic drugs for treating a disease, the method implemented by a computer processor executing program instructions comprising:
a. identifying a genetic interaction graph according to the method described above;
b. Incising pairs from said genetic interaction graph for which both genes are known drug targets; and
c. Prioritizing said pairs found in step (b) according to in-vitro experiments.

In one embodiment, the disease comprises cancer. In another embodiment, the cancer is solid cancer or hematological cancer. In another embodiment, the solid cancer is selected from breast cancer, gastrointestinal cancer, skin cancer, lung cancer, brain cancer, bladder cancer, cervical cancer, endometrial cancer, kidney cancer, lip cancer, oral cancer, liver cancer, ovarian cancer, pancreatic cancer, prostate cancer, thyroid cancer or any combination thereof. In another embodiment, the hematological cancer is selected from Leukemia, Non-Hodgkin Lymphoma, Hodgkin Lymphoma, Multiple Myeloma or any combination thereof.

In some embodiments, treating a disease comprises treating a tumor. In one embodiment, treating a tumor comprises decreasing the size of the tumor. In one embodiment, treating a tumor comprises eliciting an enhanced immune response against the tumor. In one embodiment, treating a tumor comprises delaying metastasis. In one embodiment, treating a tumor comprises increasing survival of a patient. In one embodiment, treating a tumor comprises increasing the relapse time, or disease free survival (DFS) time. In one embodiment, treating a tumor comprises increasing progression free survival (PFS) time. In one embodiment, treating a tumor comprises increasing the quality of life of a patient.

In one embodiment, the present disclosure relates to method for designing a clinical trial for a therapy, the method implemented by a computer processor executing program instructions comprising:
a. stratifying a population of patients according to the method described in the method described above; and
b. including in said clinical trial only patients predicted to be responsive to the therapy.

In one embodiment, the present disclosure relates to a method for prioritizing in-vitro models for drug development, the method implemented by a computer processor executing program instructions comprising: the method according to the method described above, where the stratification is done for cell-lines instead of human patients.

In one embodiment, the present disclosure relates to a method for repurposing existing drugs to novel indications, wherein a given drug targets a given gene or a given set of genes, the method implemented by a computer processor executing program instructions comprising:
a. Stratifying patient cohorts from different cancer types according to the method described above; and
b. Identifying cohorts with a maximal number of predicted responders.

In one embodiment, the population of samples comprises human cells, patients, or combination thereof. In another embodiment, the population comprises human cells. In another embodiment, the population comprises patients. In another embodiment, the population comprises patients diagnosed with cancer. In one embodiment, the cancer is solid cancer or hematological cancer. In another embodiment, the solid cancer is selected from breast cancer, gastrointestinal cancer, skin cancer, lung cancer, brain cancer, bladder cancer, cervical cancer, endometrial cancer, kidney cancer, lip cancer, oral cancer, liver cancer, ovarian cancer, pancreatic cancer, prostate cancer, thyroid cancer or any combination thereof, in another embodiment, the hematological cancer is selected from leukemia, Non-Hodgkin lymphoma, Hodgkin lymphoma, Multiple myeloma or any combination thereof.

### EXAMPLES

### Example 1

The depletion test fits a modified Gumbel model to the joint distributions of gene pairs based on genomic data such as mRNA expressions or copy number variations taken from a population of cancer patients. In the example below, each individual is represented by a vector of numerical values representing the genomic data for each gene in the genome. Taking a population of patients, a distribution for each gene can be built. As this distribution is not uniform by default, in order to meet the requirement for uniform marginal distributions, every gene distribution was transformed by ranking its values such that the lowest value is given the lowest rank and so on (ranks are in range [0,1]). This enforces every transformed distribution to be uniform, hence the Gumbel model is applicable to the joint transformed distributions of any pair of genes.

In the next step, the theta value that maximizes the likelihood of the modified Gumbel model was calculated using maximum likelihood estimation. Confidence rays can be calculated to identify theta values indicating a strong level of depletion near the origin of the joint distribution. This theta value was also used later on to identify synthetic lethal interactions.

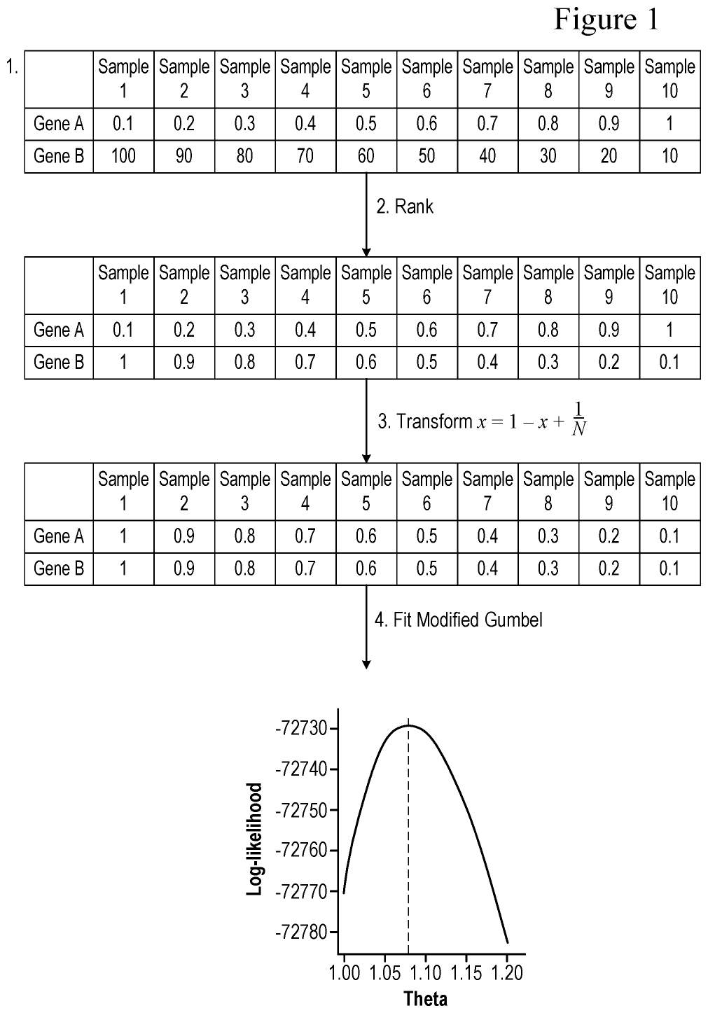

FIG. 1 illustrates the below described test algorithm—
a) The input for the depletion test is comprised of genomic data of a gene pair across a population of N samples (e.g. patients).
b) To make the distribution of each gene compliant with the Gumbel model, each distribution was ranked and divided by the number of samples to obtain values in the range [0,1].
c) To account for depletion near the origin, all values of Gene A were transformed: x⇒

$$1 - x + \frac{1}{N} .$$

d) Using these transformed distributions, the theta value was calculated, which maximizes the likelihood of the Gumbel model. Namely, θ\hat{ } was calculated such that:

$${θ}^{⋀} = {argmax}_{θ} {\sum }_{i = 1}^{n} log [ {Gumbel}_{θ} ( {g}_{1 i} , {g}_{2 i} ) ]$$

wherein Gumbel_{θ} denotes the Gumbel density function induced by θ and gⱼᵢ denotes the value of gene j in patient i.

The parabola-shaped function shown in FIG. 1 describes the trajectory of summed log-likelihood values as a function of theta for an example gene pair. The vertical dashed line marks θ\hat{ }.

### Example 2

To test whether the new depletion model based on Gumbel copula outperforms the test based on the SLIDE 1.0 hypergeometric test, a list of 2229 gene pairs known to be synthetically lethal was composed according to the scientific literature (termed Gold standard) and the power of both methods was tested in detecting them.

To do so, both tests ran on 100,000 random pairs and obtained the empirical distributions of their respective results. Next, the percentiles for each random distribution were calculated.

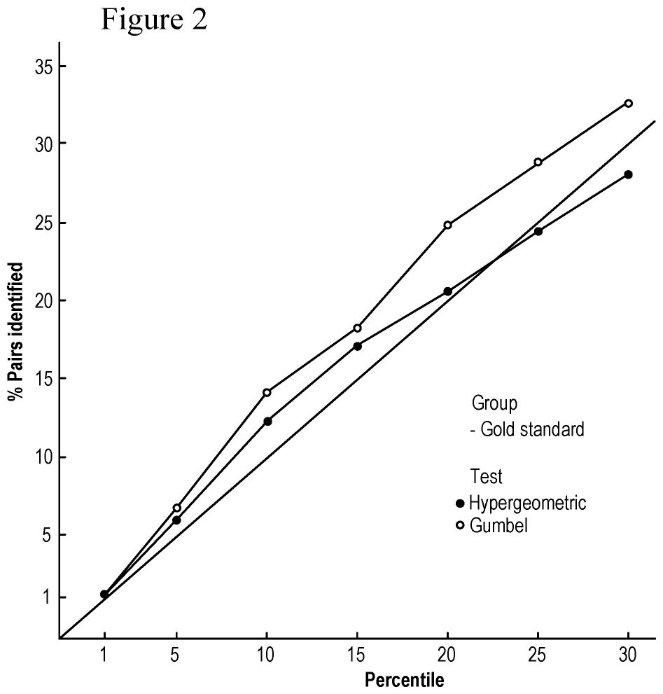

As can be seen in FIG. 2, the results show the percentage of pairs (Y-axis) that can be identified at a significant level marked on the X-axis based on the respective random distribution. Results for the Gumbel method were colored blue while the results of the hypergeometric test were colored red. The black solid line denotes the expected percent of pairs identified for each significant level, under the null hypothesis. Overall, the Gumbel method identified known GIs to a larger extent than the hypergeometric method, throughout a wide threshold range.

### Example 3

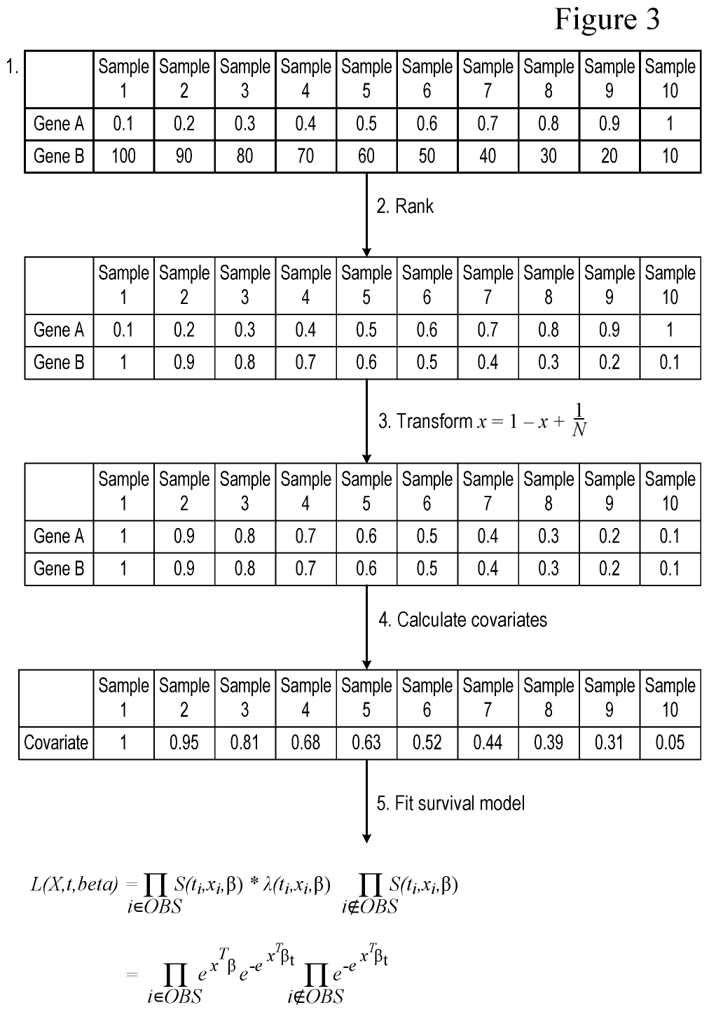

FIG. 3 demonstrates the below described survival test algorithm—
1. The input for the survival test is comprised of genomic data of a gene pair across a population of N samples (e.g., patients).
2-3. The calculation of the covariate which expresses the simultaneous inactivation state relies on the Gumbel distribution described above. The values of each gene were normalized and transform as described in steps 2-3 of the depletion test to comply with the Gumbel distribution.
4. Given the transformed values, the covariate expressing the simultaneous inactivation state was calculated as follows: let Gumbel_{θ\hat{ }} be the Gumbel model that best fits the joint distribution of the pairs as explained above. The covariate for patient i is:

$${x}_{i , covariate} = log ( \frac{max ( {Gumbel}_{{θ}^{⋀}} )}{{Gumbel}_{{θ}^{⋀}} ( {g}_{1 i} , {g}_{2 i} )} )$$

Where max(Gumbele_{θ\hat{ }}) is the maximal value of the density function Gumbel_{θ\hat{ }}
5. Let X∈R^{n,k} be a covariate matrix of n patients, each contains k characteristics such as the covariate calculated in step 4, age, gender, cancer stage etc. Let t∈Rⁿ be the times until failure or censoring for each patient and let β∈R^{k} be the coefficient vector associated with each of the k characteristics.
The likelihood of the parametric survival model given X, t, beta is:

$${x}_{i , covariate} = log ( \frac{max ( {Gumbel}_{{θ}^{⋀}} )}{{Gumbel}_{{θ}^{⋀}} ( {g}_{1 i} , {g}_{2 i} )} ) \\ {\prod }_{i ∈ OBS} {e}^{{x}^{T} β} {e}^{- {e}^{{x}^{T} {β}_{t}}} {\prod }_{i ∉ OBS} {e}^{- {e}^{{x}^{T} {β}_{t}}}$$

Where S is the survival function, λ is the hazard function, xᵢ is the covariate vector for patient i and OBS are the group of patients that were not censored (i.e. they were deceased during follow-up). The maximum likelihood estimators (MLE) for β can be calculated numerically using the Newton-Raphson method for finding extremum points of a function (see Kendall E. Atkinson, An Introduction to Numerical Analysis, (1989) John Wiley & Sons, Inc, ISBN 0-471-62489-6, incorporated herein by reference).
The MLE is important for the coefficient of the simultaneous activation state βₛₐₛ\hat{ }. This coefficient determines whether the simultaneous inactivation of the gene pair is associated with better survival. P-value can be calculated for the coefficient using the fisher information matrix of the coefficients.
   Finally, βₛₐₛ\hat{ } is used at later stages to infer GIs.

### Example 4

To test whether the new parametric survival model outperforms the test based on cox regression, a list of 2229 gene pairs known to be synthetically lethal according to the scientific literature as described above was used to test the power of both methods in detecting them.

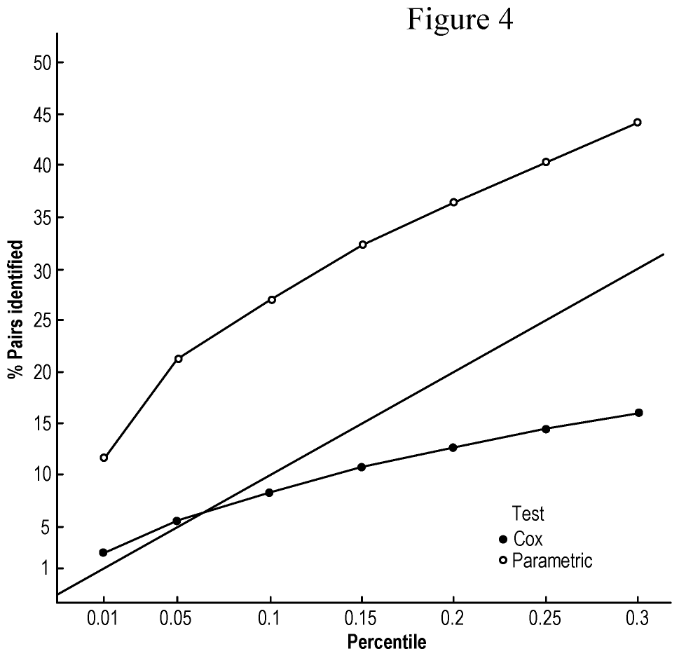

The results show the percentage of pairs (Y axis) that were identified at the significant level denoted on the X axis. Here there was no need to calculate the empirical null distribution as in example 2, since both tests have theoretical null distribution, so the P-values for each test can be directly calculated. As can be seen in FIG. 4, Results for the novel parametric method were colored blue while the results of the former cox test were colored red. The black solid line denotes the expected percent of pairs identified for each significant level under the null hypothesis. The new survival test far exceeds the performance of the cox-based model in identifying known GIs.

Next, it was tested if not only does the new parametric survival test outperform the cox-based one in identifying known GIs, but if it is also more robust to perturbations in the data. In the cox model, it was observed that even small perturbation in the data can cause the resulting coefficient (βₛₐₛ) to deviate significantly from the original one, obtained without perturbations. To check the extent of this phenomenon and compare it to the new method, the dispersion of beta values was calculated in 100 random pairs in the following manner: first, βₛₐₛ was calculated without perturbation. Then, a single perturbation in the expression data, was introduced by swapping the expression values of two randomly chosen patients, and calculate β′ₛₐₛ. This process was repeated 100 times. The absolute difference between the 100 perturbed β′ₛₐₛ and βₛₐₛ were calculated and the mean difference was recorded.

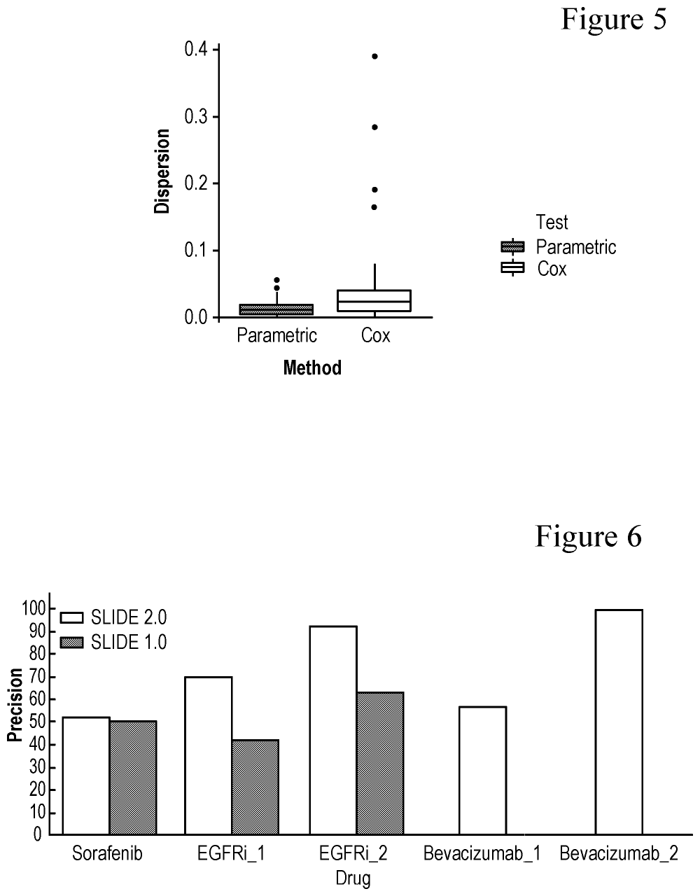

As can be seen in FIG. 5, the plot shows the distribution of mean differences (termed the “Dispersion”) over 100 random pairs for each method as boxplots. This plot demonstrates that the new parametric model is indeed more robust to perturbations, with far lower dispersion than in the cox model.

Next, response prediction for cancer patients was tested using improvements in both tests.

As can be seen in FIG. 6, The plot shows the performance of response prediction for several patient groups, each treated with a specific drug. In the case of EGFR inhibitors (EGFRi) multiple datasets targeting the same gene were merged to obtain larger sample sizes. For each group, the response to treatment was predicted for all patients based on the previous method that incorporated the old tests (SLIDE 1.0) and based on the new method which incorporated the new tests described here (SLIDE 2.0). The performance of both methods was assessed using Area Under the Curve of the Receiver Operating Characteristic curve (ROC AUC), a common measure for accuracy of predictive models.

For all cases, the predictive value of the new method was superior to the old one. In the case of the drug “Bevacizumab”, the old method did not produce any results.

### Example 5

The objective of this Example was to evaluate ENLIGHT performance in identifying the true responders among patients' cohorts, as well as identifying non-responding patients in clinical trials.

Data collection. The public domain was surveyed for available cohorts of patients receiving targeted therapies or immunotherapies, containing both pre-treatment transcriptomics and response information (either RECIST or a binary classification of response). 23 real world datasets were identified which were not previously analyzed by either SELECT or ENLIGHT, and can hence serve as unseen datasets: 22 datasets from GEO, ArrayExpress, CTRDB or the broader literature published by February 2022, and one dataset that was obtained as part of a collaboration with Massachusetts General Hospital (MGH) which we publish here for the first time. Six datasets were selected already analyzed along with two of these 23 unseen sets to serve as tuning sets. These eight tuning datasets were selected as they span a range of different treatments, therapeutic classes, response rates and sample sizes, reflecting diverse real-world data, covering five targeted therapies and one immune checkpoint blockade (ICB). These datasets were used to tune the parameters of ENLIGHT, including the GI network size and a decision threshold on the ENLIGHT Matching Score (EMS) that is used for predicting response (see below). The remaining 21 unseen datasets were set aside as unseen data for evaluation.

All datasets were coupled with response to treatment in the form of either: (i) RECIST criteria response evaluations or (ii) binary classifications of responders and non-responders that was not exclusively defined using RECIST and in several cases was not specified. In this study, we classify a patient as responder if he/she had a RECIST evaluation of CR/PR, or if he/she had a binary classification of responder. The rest were classified as non-responders. In each dataset we only analyzed patients for whom both pre-treatment transcriptomics and response data was available.

### ENLIGHT's Performance in Identifying the True Responders Among Patients' Cohorts:

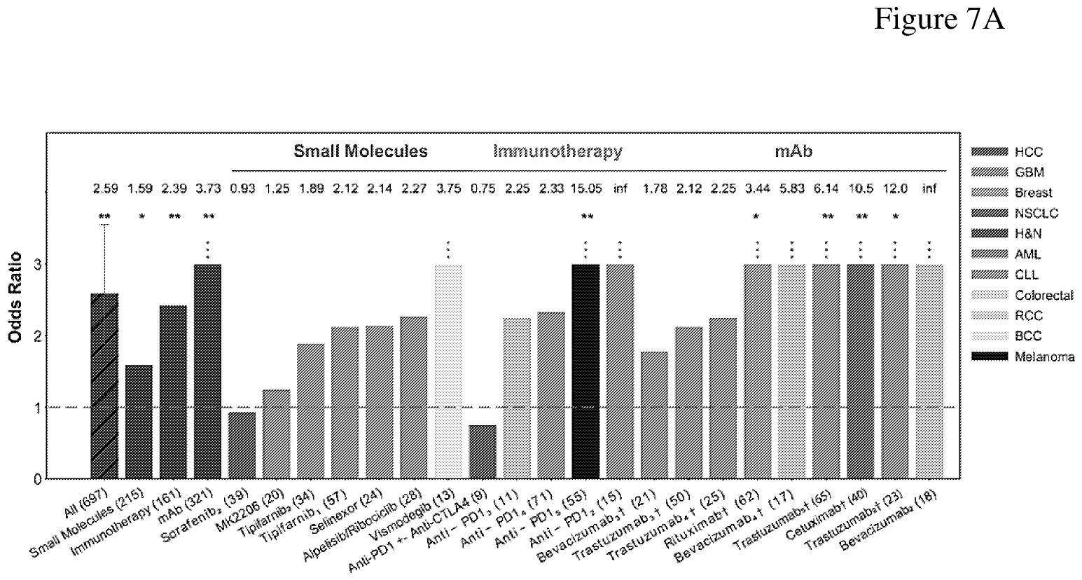

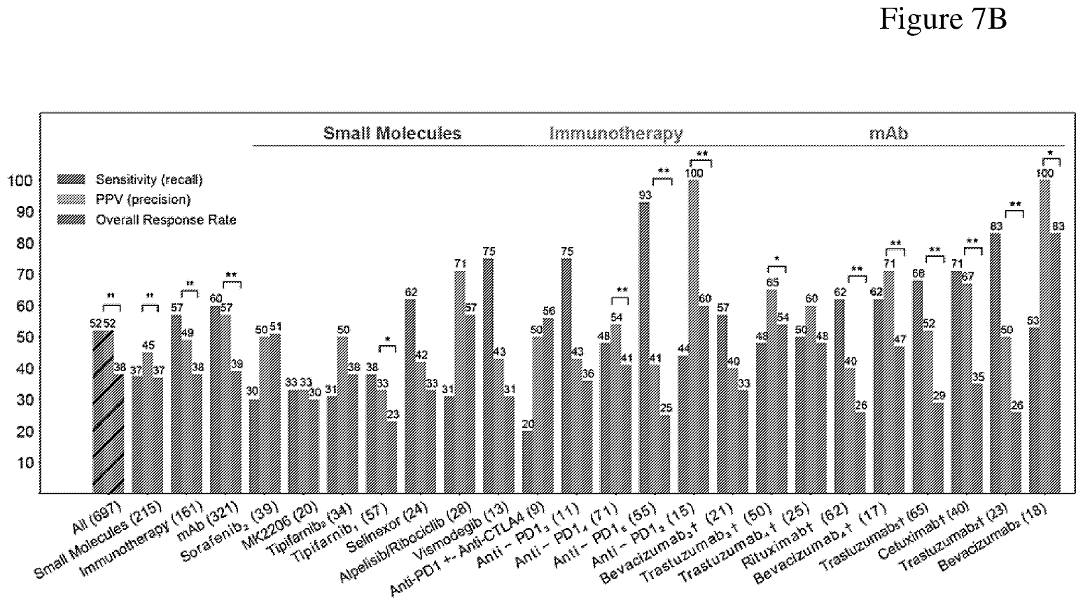

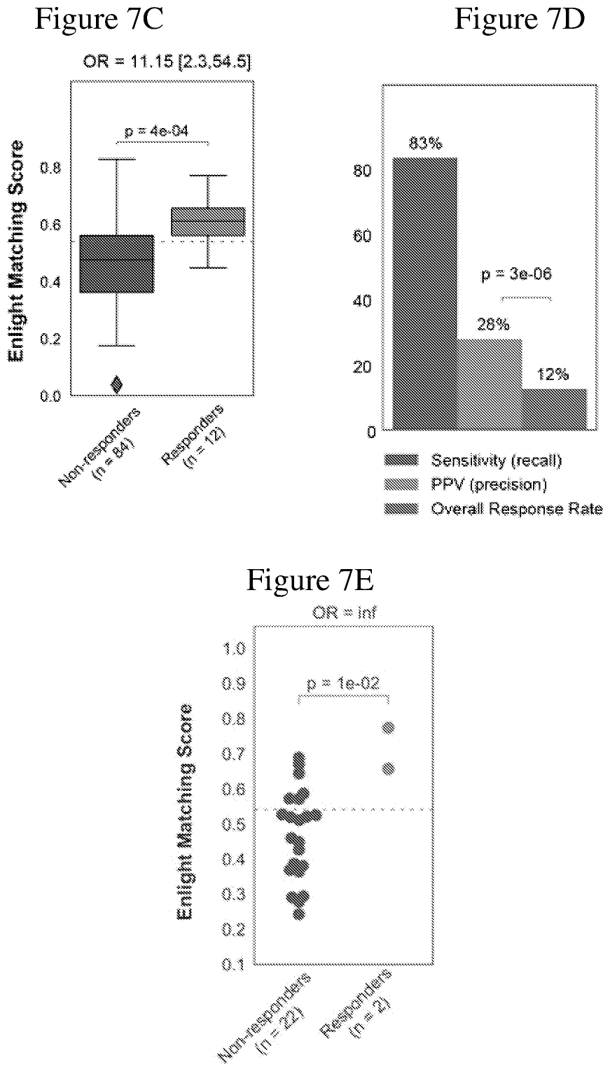

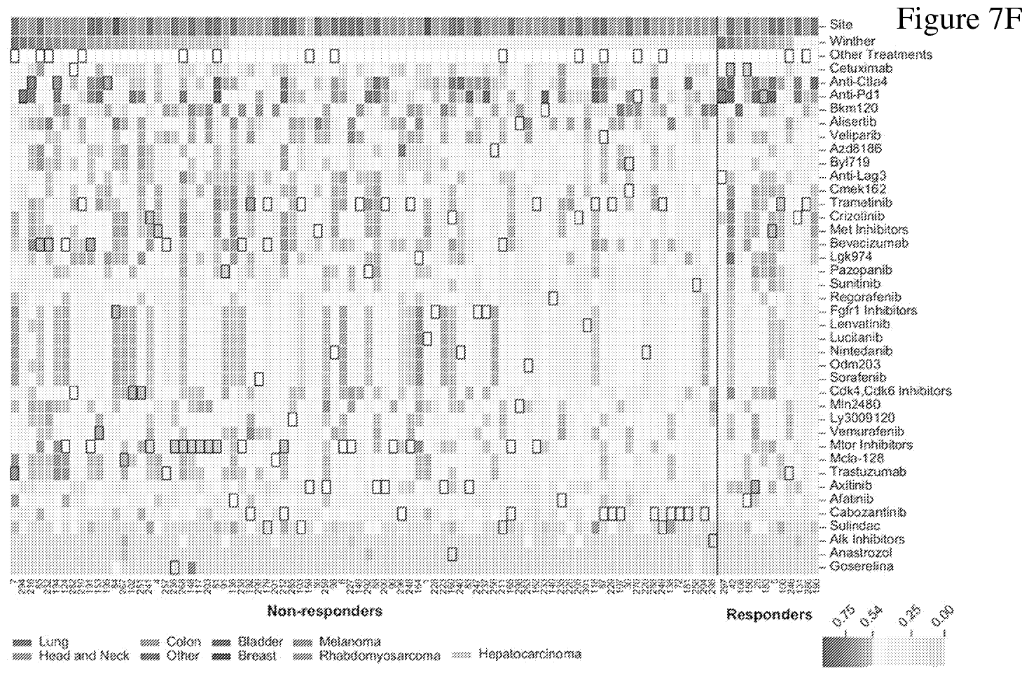

21 datasets of patient cohorts collected, spanning ICBs, mAbs, and targeted small molecules were evaluated. Notably, the response data for all evaluation cohorts was unblinded only after finalizing the ENLIGHT pipeline, including fixing the decision threshold and calculating EMS for all patients. FIG. 7A shows that ENLIGHT-matched treatments are associated with better patient response (OR>1) in all cohorts except for two (sorafenib2 and one ICB cohort), with an aggregate OR of 2.59 (95% CI, 1.89-3.55; p=3.41e-8, n=697). Correspondingly, FIG. 7B shows that the overall PPV obtained for ENLIGHT-matched cases is markedly higher than the overall response rate (52% versus 38%, a 36.84% increase, p=3.30e-13, one-sided proportion test).

Interestingly, ENLIGHT was more accurate in immunotherapies and other mAbs versus targeted small molecules, which aligns with its reliance on drugs that have accurate targets. More specifically, within the small-molecule class, ENLIGHT is only less predictive in drugs with many targets (sorafenib, a broad tyrosine kinase inhibitor and MK2206, a pan-AKT inhibitor). Notably, when a patient received a combination of targeted and chemotherapy agents (see cohorts marked with a cross), the EMS was calculated for the targeted agent alone; however, remarkably, the performance is still maintained.

In addition, ENLIGHT was evaluated as a personalized oncology tool in a multi-arm clinical trial setting, by analyzing data from the WINTHER trial, a large-scale prospective clinical trial that has incorporated genetic and transcriptomic data for cancer therapy decision making in adult patients with advanced solid tumors. ENLIGHT was able to provide predictions for all patients, except four (see STAR Methods). The EMS of the responders were significantly higher than those of non-responders (p=4e-04, FIG. 7C). The OR of ENLIGHT-matched treatments is 11.15 (p=8e-04, FIG. 7C), and the PPV is more than two times higher than the overall response rate (FIG. 7D).

Further analysis shows that responders had significantly higher EMS than non-responders also for the 24 patients treated with a combination of drugs (FIG. 7E) and that ENLIGHT-matched treatments were associated with better response, without being hampered by the background of chemotherapy treatment. FIG. 7F depicts the landscape of different treatment alternatives with high EMS scores for each patient. We observe that 91/96 patients (94.8%) had at least one treatment with which they were ENLIGHT-matched, highlighting the potential coverage of ENLIGHT in real-world cases.

### ENLIGHT's Performance in Identifying Non-Responding Patients in Clinical Trials:

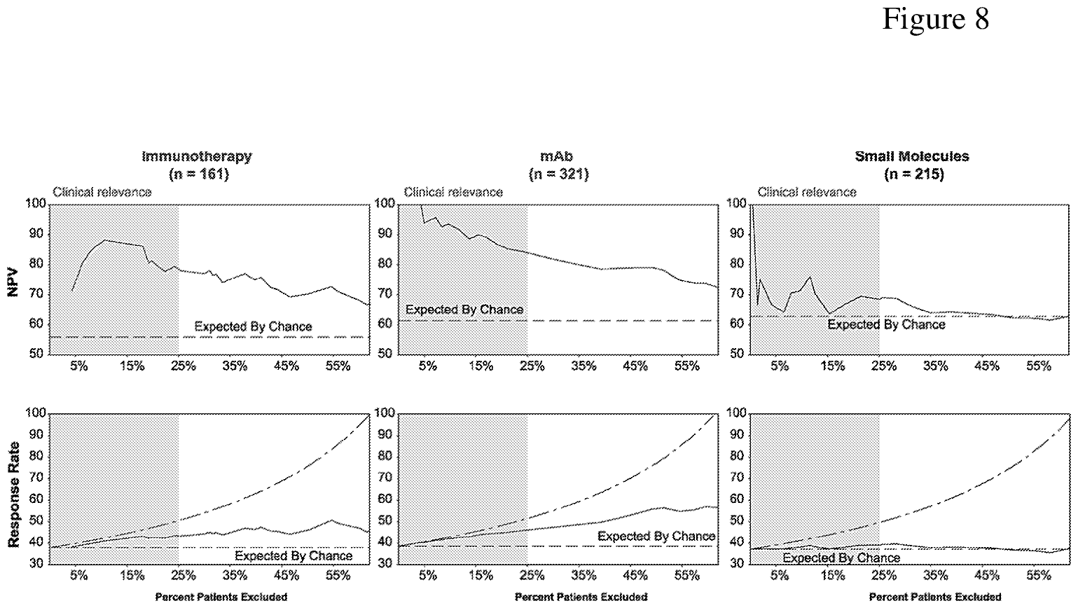

In the clinical trial design (CTD) scenario, there is a need of identifying sub-populations of non-responding patients who could be excluded from the trial a priori, thereby allowing smaller studies to achieve higher response rates with adequate statistical power. The upper row of FIG. 8 depicts the proportion of true non-responders among those predicted not to respond (NPV) as a function of the percent of patients excluded, where patients are excluded by order of increasing EMS. For both immunotherapy and other mAbs, ENLIGHT's NPV curve is considerably higher than the NPV expected by chance, i.e., the percentage of non-responders, testifying to its benefit. For targeted small molecules, however, it is unable to reliably identify non-responders, an issue that should be further studied and improved upon in future work.

The bottom row of FIG. 8 depicts the response rate in the remaining cohort after excluding patients with EMS below the decision threshold. As evident, ENLIGHT-based exclusion considerably increases the response rate among the remaining patients (middle, solid line). The dotted-dashed line represents the limit performance of an optimal “all-knowing” classifier that excludes all non-responders, retaining only true responders (correspondingly, the x axes end when this optimal classifier excludes all true non-responders, achieving the optimal response rate of 100%). Focusing on a practical exclusion range of up to 25% of patients (shaded area), ENLIGHT-based exclusion achieves 87%-97% and 90%-99% of the optimal exclusion response rate, for both immunotherapy and other mAbs, respectively (Table 1). It is important to acknowledge that the ENLIGHT-based exclusion strategy assumes knowledge of the EMS distribution in the trial, which may not be known a priori, but could be estimated using historical transcriptomics data from a reference population of the pertaining cancer indication and clinical characteristics.

TABLE 1

% patients excluded

| | | 5 | 10 | 15 | 20 | 25 |
|---|---|---|---|---|---|---|
| % of optimal | Immunotherapy | 97 | 97 | 93 | 90 | 87 |
| response rate | (n = 161) | | | | | |
| | mAb | 99 | 98 | 96 | 94 | 90 |
| | (n = 321) | | | | | |

For each percent of patient exclusion (columns), the response rate among the remaining patients when excluding based on increasing EMS is given as a percentage of the upper bound response rate achieved by the “all knowing” optimal classifier that excludes only true non-responders.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for identifying a pair of genes comprising a synthetic lethality (SL), synthetic rescue (SR), or synthetic dosage lethality (SDL) interaction, using a depletion model, said method implemented by a computer processor executing program instructions comprising:
a. Transforming expression data relating to each of two genes from a population, through ranking, thereby producing two uniform transformed distributions in the range [0, 1];
b. Calculating a resulting joint expression distribution for the gene pair, having uniform marginal distributions;
c. Identifying a parametric family of distributions comprising a shape parameter wherein said shape parameter determines the degree of corner depletion or enrichment for one or more of the corners in the joint distribution, and fitting said shape parameter to said joint expression data; and
d. Calculating a value of a best-fitting shape parameter as an indication of the genetic interaction between said two genes.

2. A method according to claim 1, wherein said synthetic rescue (SR) comprises synthetic rescue DD (SR-DD) or synthetic rescue DU (SR-DU).

3. A method according to claim 1, wherein for identifying a pair of genes comprising a synthetic lethality (SL) interaction, said shape parameter would measure depletion in the lower left corner of said joint distribution.

4. A method according to claim 2, wherein for identifying a pair of genes comprising a synthetic rescue DD (SR-DD) interaction, said shape parameter would measure enrichment in the lower left corner of said joint distribution.

5. A method according to claim 2, wherein for identifying a pair of genes comprising a synthetic rescue DU (SR-DU) interaction, said shape parameter would measure enrichment in the upper left corner of said joint distribution.

6. A method according to claim 1, wherein for identifying a pair of genes comprising a synthetic dosage lethality (SDL) interaction, said shape parameter would measure depletion in the upper left corner of said joint distribution.

7. A method according to claim 1, wherein said parametric family of distributions comprises Gumbel copulas and said shape parameter comprises a parameter theta of the copula.

8. A method according to claim 7, comprising:
a. selecting a pair of genes from genomic data across a population of N samples;
b. building a distribution for each of said pair of genes across the population;
c. for each of said pair of genes, assigning ranks corresponding to said distribution, the ranks being evenly distributed between 0 and 1, i.e., ranking the distribution of each gene and dividing by the number of samples to obtain values in the range [0,1];
d. obtaining data by mirroring said distribution horizontally, by transforming the ranks for one of the genes by:
$$x ⇒ 1 - x + \frac{1}{N} .$$
e. calculating a theta value which maximizes a likelihood of a Gumbel model of said data obtained in (d) for the gene pair:
$${θ}^{⋀} = {argmax}_{θ} {\sum }_{i = 1}^{n} log [ {Gumbel}_{θ} ( {g}_{1 i} , {g}_{2 i} ) ] .$$

9. A method for identifying a pair of genes comprising a synthetic lethality (SL), synthetic rescue (SR-DD or SR-DU), or synthetic dosage lethality (SDL) interaction, using a parametric survival model, said method implemented by a computer processor executing program instructions comprising:
a. Transforming expression data relating to each of two genes from a population, thereby producing two uniform transformed distributions in the range [0, 1];
b. Calculating a resulting joint expression distribution for the gene pair, having uniform marginal distributions;
c. Identifying a theoretical distribution function D that approximates the joint distribution of the transformed expression levels of said pair of genes;
d. Calculating a covariate value (c(p)) for a given patient in a population of patients (cohort P), by calculating a ratio of the density of said theoretical distribution function D at joint expression values (x,y) of said gene pair, to a maximal D density value or a minimal D density value, across a full joint distribution space; and
e. Assessing a correlation between (i) a set of covariates C:={c{p)|p in P} obtained in “c” for said patients of cohort P; and (ii) survival of the patients in said cohort, as an assessment of the strength of the corresponding genetic interaction between said gene pair.

10. A method according to claim 9, wherein said distribution function D is identified using a depletion model.

11. A method according to claim 9, wherein said distribution function comprises a Gumbel copula statistical model.

12. A method according to claim 11, comprising:
a. Selecting a pair of genes from genomic data across a population of N samples;
b. Building a distribution for each of said pair of genes in the population;
c. For each gene, assigning ranks corresponding to said distribution, the ranks being evenly distributed between 0 and 1, i.e., ranking the distribution of each gene and dividing by the number of samples to obtain values in the range [0,1];
d. Obtaining data by mirroring said distribution horizontally, by transforming the ranks for one of the genes by:
$$x ⇒ 1 - x + \frac{1}{N} .$$
e. Calculating theta θ;
f. For each patient p from said sample, calculating a covariate cp(p) according to:
$$c ( p ) = log ( \frac{( {Gumbel}_{{θ}^{⋀}} )}{{Gumbel}_{{θ}^{⋀}} ( {g}_{1} , {g}_{2} )} )$$
where _g(i,j) denotes the expression of gene gi in patient j
g. Calculating likelihood of the parametric survival model according to:
$$L ( C , t , beta ) = {\prod }_{i ∈ OBS} S ( {t}_{i} , {c}_{i} , β ) * λ ( {t}_{i} , {c}_{i} , β ) {\prod }_{i ∉ OBS} S ( {t}_{i} , {c}_{i} , β ) \\ {\prod }_{i ∈ OBS} {e}^{{C}^{T} β} {e}^{- {e}^{{C}^{T} {β}_{t}}} {\prod }_{i ∉ OBS} {e}^{- {e}^{{C}^{T} {β}_{t}}} .$$

13. A method according to claim 1, wherein said expression data comprises single-cell data, shRNA/sgRNA screens, or CRISPR single gene knockout, drug screens, patient data from the Cancer Genome Atlas (TCGA) or any combination thereof.

14. A method according to claim 1, wherein said distribution is measured directly through protein expression data, deduced from measurements of methylation, silencing DNA mutations, mRNA expression, mRNA copy number variation or any combination thereof.

15. A method according to claim 1, wherein said population comprises human cell lines, patients, or combination thereof.

16. A method for identifying a pair of genes comprising a synthetic lethality (SL), synthetic rescue (SR-DD or SR-DU), or synthetic dosage lethality (SDL) interaction, the method implemented by a computer processor executing program instructions comprising combining a depletion model and a parametric survival model.

17. A method for creating genetic interaction graphs, said method implemented by a computer processor executing program instructions comprising
a. method for identifying a pair of genes comprising a synthetic lethality (SL), synthetic rescue (SR-DD or SR-DU), or synthetic dosage lethality (SDL) interaction;
b. including in the interaction graph all the genes belonging to gene pairs that passed the identification of step (a); and
c. marking each of said gene pairs with the type of interaction identified for it (namely, one of SL, SR-DD, SR-DU or SDL).

18. A method of predicting responsiveness of a patient to a therapy targeting a set of target genes, the method implemented by a computer processor executing program instructions comprising:
a) identifying a genetic interaction graph according to claim 17,
b) incising from said genetic interaction graph of step (a) a sub-network comprising said target genes and all other genes connected to said target genes (“partner genes”)
c) determining the activity of each said partner gene paired with one of the target genes, wherein low activity of multiple SL, SDL or SR-DU partner genes, and/or high activity of multiple SR-DD partner genes is indicative of high responsiveness to the therapy targeting said target genes;
thereby predicting the responsiveness of the patient to the therapy.

19. A method of stratifying a population of patients according to the responsiveness to a therapy targeting a set of target genes, the method implemented by a computer processor executing program instructions comprising:
a. predicting responsiveness of each patient of the population to the therapy according to the method described in claim 18;
b. stratifying said population of patients according to their responsiveness to the therapy.

20. A method for identifying a drug target for a disease, wherein the disease is associated with the inactivation of a single target gene, the method implemented by a computer processor executing program instructions comprising:
a. identifying a genetic interaction graph spanning the target genes according to claim 17;
b. incising from said genetic interaction graph of step (a) a sub-network comprising said target gene and all other genes connected to said target gene;
c. for each said target gene, stratifying designated patient population cohort P, according to claim 19, based on predicted response to inhibition of said target gene; and
d. identifying the most attractive potential drug targets, as those target genes for which a significant sub-population of patients is expected to respond to target inhibition.

21. A method for identifying synergistic drugs for treating a disease, the method implemented by a computer processor executing program instructions comprising:
a) identifying a genetic interaction graph according to claim 17;
b) Incising pairs from said genetic interaction graph for which both genes are known drug targets; and
c) Prioritizing said pairs found in step (b) according to in-vitro experiments.

22. A method for designing a clinical trial for a therapy, the method implemented by a computer processor executing program instructions comprising:
a) stratifying a population of patients according to the method described in claim 19;
b) including in said clinical trial only patients predicted to be responsive to the therapy.

23. A method for prioritizing in vitro models for drug development, the method implemented by a computer processor executing program instructions comprising: the method according to claim 19, where the stratification is done for cell-lines instead of human patients.

24. A method for repurposing existing drugs to novel indications, wherein a given drug targets a given gene or a given set of genes, the method implemented by a computer processor executing program instructions comprising:
a) Stratifying patient cohorts from different cancer types according to claim 19.
b) Identifying cohorts with maximal number of predicted responders.

25. A method for expanding the indications of a drug targeting a gene or a set of genes, the method implemented by a computer processor executing program instructions comprising:
a) providing a cohort of patients having a medical condition, wherein said condition is not indicated to said drug;
b) predicting the responsiveness of each patient of said cohort to a therapy comprising administering said drug, according to the method of claim 18;
wherein high responsiveness to said therapy indicates that said drug can be indicated to said medical condition.

26. A method for expanding the indications of a drug targeting a gene or a set of genes, the method implemented by a computer processor executing program instructions comprising:
a) providing a group of patients having a medical condition, wherein said condition is not indicated to said drug;
b) predicting the responsiveness of each patient to a therapy comprising administering said drug, according to the method of claim 18;
c) stratifying said patients according to their responsiveness to said therapy;
d) identifying a cohort with the maximal number of predicted responders;
wherein high responsiveness to said therapy in said cohort indicates that said drug can be indicated to said medical condition for patients belonging to said cohort.
